(19) United States
(12) Patent Application Publication
(10) Pub. No.: US 2018/0051084 A1
(43) Pub. Date: Feb. 22, 2018

(54) COMBINATION OF BETA-ADRENERGIC RECEPTOR ANTAGONISTS AND CHECK POINT INHIBITORS FOR IMPROVED EFFICACY AGAINST CANCER

(71) Applicant: Health Research, Inc., Buffalo, NY (US)

(72) Inventors: Elizabeth REPASKY, Buffalo, NY (US); Bonnie HYLANDER, Buffalo, NY (US)

(21) Appl. No.: 15/557,211

(22) PCT Filed: Mar. 14, 2016
(86) PCT No.: PCT/US16/22277

§ 371 Date: Sep. 11, 2017

Related U.S. Application Data
(60) Provisional application No. 62/132,286, filed on Mar. 12, 2015.

(51) Int. Cl. C07K 16/28; A61K 31/138; A61K 38/17; A61K 39/04; A61K 39/395; A61K 45/06

(52) U.S. Cl. CPC C07K 16/2818; A61K 31/138; A61K 38/17; A61K 2039/505; A61K 39/3955; A61K 45/06; A61K 39/04

(57) ABSTRACT

Provided are methods for prophylaxis and/or therapy of cancer that include administering to an individual in need thereof an effective amount of a β-blocker and an immune checkpoint inhibitor such that growth of cancer in the individual is inhibited. Patients include those diagnosed with or at risk for a wide variety of cancer types. Methods are provided for cancer treatment in individuals who are resistant to checkpoint inhibitor monotherapies. Greater than additive anti-cancer effects may be achieved.

### CROSS REFERENCE TO RELATED APPLICATIONS

This application claims priority to U.S. patent application No. 62/132,286, filed Mar. 12, 2015, the disclosure of which is incorporated herein by reference.

### FIELD

The present disclosure relates generally to anti-cancer approaches and more specifically to combinations of β-adrenergic receptor antagonists and checkpoint inhibitors.

### BACKGROUND

There is much evidence that patients are able to mount an immune response against their own tumors, however tumors often remain unharmed because of their ability to actively suppress, or even kill, immune cells. There are many approaches being developed to overcome this suppression and develop effective immunotherapies for cancer patients. Among the most promising are vaccines and adoptive T-cell transfer. However, these therapies are also vulnerable to tumor mechanisms of immunosuppression. Recently, it has been found that tumors employ strategies which mimic the natural mechanisms by which the immune response is down-regulated, specifically by expression of so called immune checkpoint ligands which interact with receptors on immune cells to block their activity. “Immune checkpoint inhibitors” have been developed to block these interactions. One such example of these inhibitors are antibodies to PD-1 (programmed cell death-1), a receptor on the T-cells which when bound by PD-L1 (a molecule often expressed on tumor cells) inhibits the activity of the T-cells and suppresses the anti-tumor immune response. However, there is an ongoing need for compositions and methods that can enhance the efficacy of check point inhibitors. The present disclosure relates to this need.

### SUMMARY

The present disclosure relates generally to methods for prophylaxis and/or therapy of cancer. The method comprises administering to an individual in need thereof an effective amount of a β-blocker and an immune checkpoint inhibitor such that growth of cancer in the individual is inhibited.

In certain approaches the individual is diagnosed as, or is suspected of having, or develops a cancer that is resistant to an immune checkpoint inhibitor. In certain implementations the individual is not being treated, and/or has not previously been treated with a β-blocker. In certain approaches methods of this disclosure comprise selecting an individual who has a cancer that exhibits resistance to treatment with a checkpoint inhibitor, wherein the individual is not treated with a β-blocker when the resistance is exhibited, and administering to the individual the checkpoint inhibitor and the β-blocker such that growth of the cancer is inhibited.

Methods of the disclosure can be performed using any suitable checkpoint inhibitors and β-blockers, and can include using more than one of either of these types of agents. In particular and non-limiting examples the immune checkpoint inhibitor is selected from anti-programmed cell death protein 1 (PD-1) antibody or PD-1 binding fragment thereof, or an anti-PD-1 principal ligand (PD-L1) antibody or PD-L1 binding fragment thereof. PD-1 and PD-L1 are well characterized in the art.

In certain implementations the β-blocker comprises a nonselective β-blocker and thus is pertinent to the three presently known types of beta receptors (β1, β2 and β3 receptors). In certain examples the β-blocker interferes with or more of these receptors binding to their endogenous ligands and thus may be competitive antagonists for any β-adrenergic receptor(s).

In certain approaches administering the β-blocker and the immune checkpoint inhibitor results in a greater than additive inhibition of growth of the cancer.

In certain approaches the individual treated with a combination of this disclosure has a cancer that was treated with the checkpoint inhibitor without the β-blocker, wherein the cancer was resistant to the checkpoint inhibitor, and wherein the β-blocker and the immune checkpoint inhibitor are subsequently administered to the individual such that growth of the cancer is inhibited.

In one aspect the disclosure provides a method comprising selecting an individual who has a cancer that exhibits resistance to treatment with a checkpoint inhibitor, wherein the individual is not treated with a β-blocker when the resistance is exhibited, and administering to the individual the checkpoint inhibitor and the β-blocker such that growth of the cancer is inhibited.

### BRIEF DESCRIPTION OF FIGURES

FIG. 1. Graph of data obtained using Pan02 pancreatic cell line tumors in C57BL/6 mice. Treatment of the mice with the β-blocker propranolol significantly improved the efficacy of anti-PD-1 checkpoint inhibition. *p<0.05.

FIG. 2. Graph of data demonstrating that β-AR blockade improves the efficacy of anti-PD-1 immunotherapy in a breast cancer model. Data depict absolute tumor volume. Neither propranolol nor anti-PD-1 mAb alone has any impact on tumor growth. But a combination of β-AR antagonist and checkpoint inhibitor results in a greater than additive reduction in absolute tumor volume growth.

FIG. 3. Graph of data demonstrating that β-AR blockade improves the efficacy of anti-PD-1 immunotherapy in a breast cancer model. Data depict relative tumor volume.

FIGS. 4A and 4B represent data obtained in a melanoma model. Individual tumor growth curves (FIG. 4A) and tumor growth rates (FIG. 44B) show a combination of anti-PD-1 and β-blocker is effective in melanoma tumors that do not respond to anti-PD-1 as a monotherapy.

### DESCRIPTION

The disclosure relates to methods for cancer therapy comprising administering to an individual in need a combination of an immune checkpoint inhibitor and one or more β-blockers.

In embodiments, the individual in need of treatment in accordance with this disclosure is any mammal, including but not limited to a human. The cancer type is not particularly limited, other than being a cancer type for which immune checkpoint inhibition may be a suitable prophylactic and/or therapeutic approach. In embodiments, the individual is at risk for, is suspected of having, or has been diagnosed with a cancer. In embodiments, the cancer is lung, colon, breast, pancreatic, brain, liver, bladder, kidney, melanoma, ovary, testicular, esophageal, gastric, fibrosarcoma, rhabdomyosarcoma, head and neck, renal cell, thyroid, or a blood cancer.

The disclosure is also pertinent to approaching cancers that are or may become resistant to treatment with one or more immune checkpoint inhibitors. Thus in certain implementations the individual has been previously treated for cancer with a checkpoint inhibitor, but was not given any β-blockers while being treated with the checkpoint inhibitor, and the cancer was initially resistant, or develops resistance, to the checkpoint inhibitor treatment. The disclosure thus includes selecting an individual who has cancer that is resistant to a checkpoint inhibitor as a monotherapy, and administering to the individual a checkpoint inhibitor and a β-blocker. The individual who is resistant to a checkpoint inhibitor as a monotherapy accordingly means an individual who was administered a checkpoint inhibitor for the cancer, but was not also administered a β-blocker, and the cancer was resistant to the treatment that included the checkpoint inhibitor but not the β-blocker. The monotherapy may have included other anti-cancer agents or other interventions, so long as such other agents did not include the β-blocker that is subsequently used in a combination therapy of this disclosure. In one embodiment the individual who is treated with a combination approach described herein has never been previously treated with a β-blocker. In certain embodiments the individual who is treated with a combination therapy of this disclosure has not been diagnosed with, or is not suspected of having, or is not a risk for developing a non-cancerous condition for which a β-blocker would ordinarily be prescribed.

In certain embodiments a combination of an immune checkpoint inhibitor and a β-blocker exerts a synergistic effect against cancer, which may comprise but is not limited to a greater than additive inhibition of cancer progression, and/or a greater than additive inhibition of an increase in tumor volume, and/or a reduction in tumor volume, and/or a reduction in tumor growth rate, and/or an eradication of a tumor and/or cancer cells. The method may also result in a prolonging of the survival of the individual.

The disclosure also comprises monitoring the treatment of an individual who is receiving a combination of an immune checkpoint inhibitor and a β-blocker. This approach comprises administering the combination of an immune checkpoint inhibitor and a β-blocker as a cancer treatment, testing the individual and/or a biological sample from the individual to determine the efficacy of the combination therapy, and if determined to be necessary, adjusting the combination therapy by, for example, changing the amount of the immune checkpoint inhibitor or the β-blocker, or both, and/or changing the type of immune checkpoint inhibitor and or the β-blocker. Retesting and changing the combination therapy may also be performed.

As is known in the art, β-blockers comprise a class of drug compounds that are typically used for management of cardiac arrhythmias, inhibition of secondary myocardial infarction, management of hypertension, and other indications. The present disclosure includes using any one or any combination of β-blockers that are selective or non-selective antagonists of any one or any combination of the three presently known types of beta receptors (β1, β2 and β3 receptors), or otherwise interfere with one or more of these receptors binding to their endogenous ligands, i.e., epinephrine and/or other stress hormones. Thus, in embodiments, β-blockers used in this disclosure comprises a class of competitive antagonists for β-adrenergic receptors. In one embodiment, the β-blocker is a nonselective β-blocker, such as a sympatholytic β-blocker. In one embodiment, the β-blocker is propranolol. In certain embodiments the β-blocker is selected from Bucindolol, Carteolol, Carvedilol, Labetalol, Nadolol, Oxprenolol, Penbutolol, Pindolol, Sotalol, and Timolol.

The immune checkpoint inhibitor used in combination with the one or more β-blockers can be any immune checkpoint inhibitor. As is known in the art, an example of an immune checkpoint is the transmembrane programmed cell death 1 protein (PDCD1, PD-1; also known as CD279) and its ligand, PD-1 ligand 1 (PD-L1, CD274). In normal, non-malignant physiology, PD-L1 on the surface of a cell binds to PD1 on the surface of an immune cell, which inhibits the activity of the immune cell. PD-L1 up-regulation on cancer cell surfaces is thought to facilitate evasion of the host immune system, at least in part by inhibiting T cells that would otherwise target the tumor cell. In alternative embodiments, other immune checkpoints can be inhibited, such CTLA-4.

In embodiments, any one or more checkpoint inhibitors can be combined with any one or more β-blockers for use in the methods of this disclosure. In certain embodiments, the checkpoint inhibitors that are combined with the β-blockers comprise antibodies that bind to PD-1, or anti-PD-L1, such as Nivolumab. In another embodiment, the checkpoint inhibitor is an antibody that targets CTLA-4, such as Ipilimumab. In another embodiment the checkpoint inhibitor is targets CD366 (Tim-3), which is a transmembrane protein also known as T cell immunoglobulin and mucin domain containing protein-3.

In alternative embodiments, the checkpoint inhibitors comprise small molecules or other agents that disrupt the immune checkpoint that is exploited by cancer cells to evade cell-mediated or other immune-mediated targeting.

Those skilled in the art, given the benefit of the present disclosure, will recognize how to determine an effective amount of the combination of checkpoint inhibitor and β-blocker for treatment of cancer. In general, and without intending to be bound by any particular theory, it is expected that the amounts of each of these agents that are used and/or tested currently in humans for their separate indications will also be effective in the presently provided combination approach. But modifications can be made by medical professionals based on known conditions, such as the size, age, gender and overall health profile of the individual, the type and stage of the cancer, and other conditions and risk factors that will be otherwise apparent to those skilled in the art. In embodiments, administering the checkpoint inhibitor and the β-blocker has a greater than additive effect on tumor inhibition, relative to use of either agent alone. A greater than additive effect can be determined by comparing the effects of one or both of the agents to any suitable reference, including but not limited to a predetermined value.

In embodiments, one or more β-blockers and one or more immune checkpoint inhibitors are administered concurrently. In embodiments, the one or more β-blockers and one or more immune checkpoint inhibitors are combined into a single pharmaceutical formulation. In embodiments, the one or more β-blockers and the one or more immune checkpoint inhibitors are administered sequentially. The β-blocker and immune checkpoint inhibitor can be administered via any suitable route, including but not necessarily limited to intravenous, intramuscular, subcutaneous, oral, and parenteral routes.

In an embodiment, the combination therapy has a greater than additive inhibition of tumor growth, which may be determined using any suitable measurement, non-limiting examples of which include determining tumor volume or tumor growth rate. The combination therapy can be combined with any other, conventional cancer therapies, including but not limited to surgical and chemotherapeutic approaches.

The following specific examples are provided to illustrate the invention, but are not intended to be limiting in any way.

### Example 1

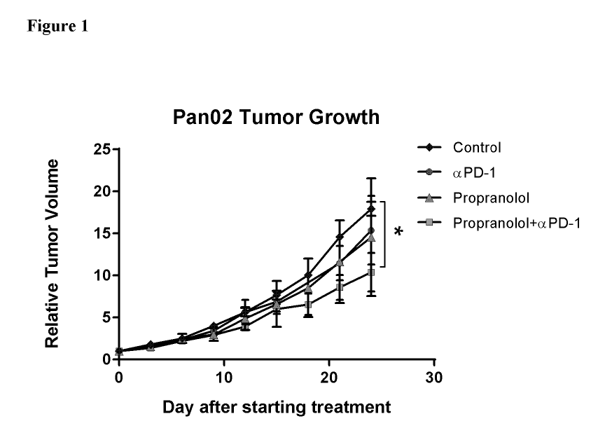

This Example shows that a combination of a β-blocker and an immune checkpoint inhibitor inhibits tumor growth, and demonstrates that the combination is capable of eliciting a greater than additive inhibition of tumor growth. In particular, and without intending to be constrained by any particular, theory, it is considered that combining specific blockade of a stress response with a β-blocker will reverse the systemic immunosuppression caused by stress, such as cancer, and when given in combination with a immune checkpoint inhibitor, will result in both improved activation (i.e., effect of the β-blocker) and sustained activity (i.e., effect of the checkpoint inhibitor) of immune cells and result in significantly improved efficacy of immune cells against the tumor. Accordingly, as shown in FIG. 1, β-AR blockade improves the efficacy of anti-PD-1 immunotherapy in a pancreas cancer model. To obtain the data presented in FIG. 1, a murine pancreatic tumor (Pan02) was engrafted in C57BL/6 mice. When tumors reached an average size of 50-100 mm³, tumor bearing mice were assigned to one of four experimental groups and treatment was initiated. Groups of mice received: (1) saline (control), (2) anti-PD-1 antibody (BioExcel, 200 μg/dose every 3 days), (3) the β-adrenergic signaling antagonist propranolol (10 mg/kg daily by intraperitoneal injection) or (4) combination therapy. Tumor growth was monitored and graphed as relative tumor volume compared to each tumor's starting volume. As can be seen in FIG. 1, neither anti-PD-1 nor propranolol alone caused a statistically significant decrease in tumor growth. However, tumor growth was statistically significantly slowed in mice receiving the combination therapy (Students t-test) such that the effect was greater than additive. The data accordingly indicate that the combination therapy can be effective against pancreatic tumors that are resistant to treatment with the checkpoint inhibitor alone.

### Example 2

This Example demonstrates that β-AR blockade improves the efficacy of anti-PD-1 immunotherapy in a breast cancer model. These data, and those obtained using a B16 tumor data provided below were obtained as described for FIG. 1, except β-AR blockade (the propranolol treatment) was started three days before administering the anti-PD-1 checkpoint inhibitor, and this 4T1breast cancer model used BALB/c mice.

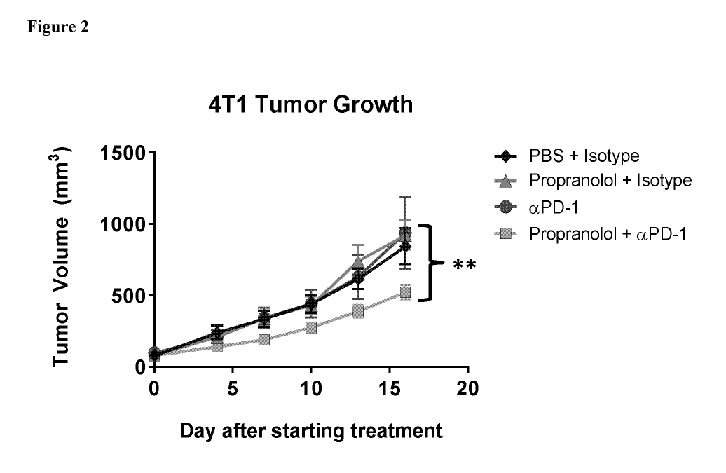

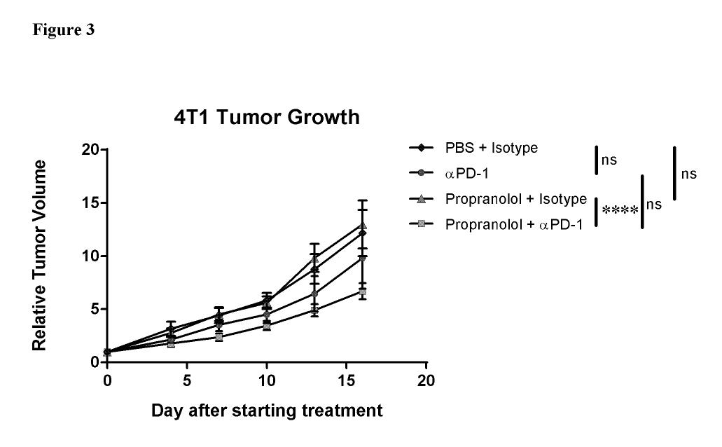

As can be seen from FIGS. 2 and 3 (measuring absolute tumor volume and relative tumor volume, respectively), neither propranolol nor anti-PD-1 mAb alone has any impact on tumor growth. But a combination of β-AR antagonist and checkpoint inhibitor results in a greater than additive reduction in absolute tumor volume growth. Thus, as with the pancreatic cancer model, the data demonstrate that this combination approach is effective even in breast cancer tumors that do not respond to anti-PD-1 as a monotherapy.

### Example 3

This Example demonstrates that β-AR blockade improves the efficacy of anti-PD-1 immunotherapy in a melanoma model.

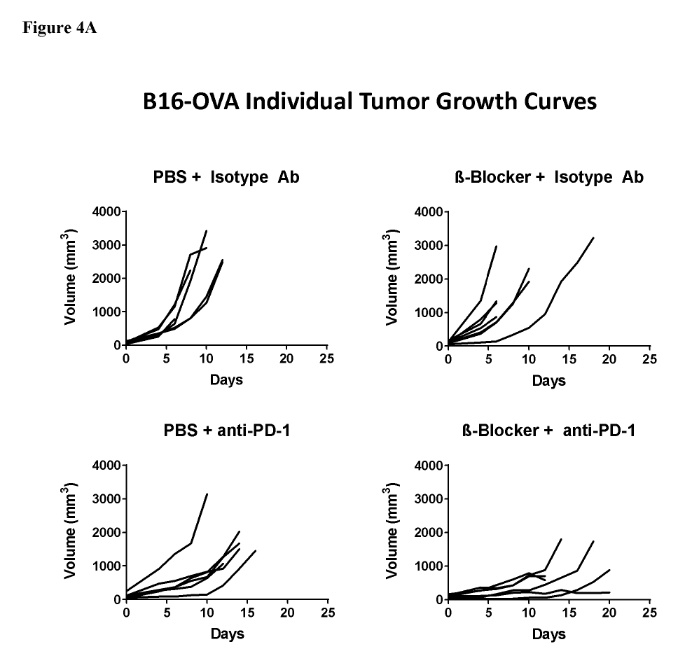

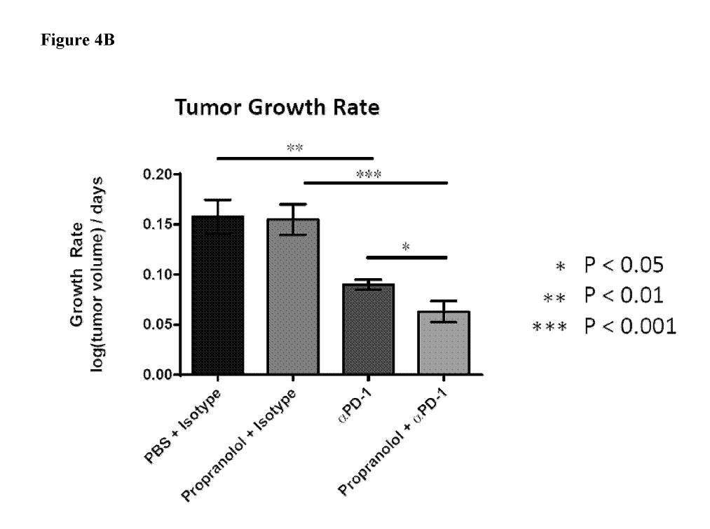

As can be seen from FIGS. 4A and 4B, which summarize data reflecting individual tumor growth curves (FIG. 4A) and tumor growth rates (FIG. 4B), the combination approach is effective even in melanoma tumors that do not respond to anti-PD-1 as a monotherapy.

While the invention has been described through specific embodiments, routine modifications will be apparent to those skilled in the art and such modifications are intended to be within the scope of the present invention. But as can be seen from the foregoing examples, and again without intending to be constrained by any particular theory, it is believed that the presently described combination approach is superior to the use of either single agent because the β-blocker will act to reverse the high levels of immunosuppressive cells (MDSC and T-regs) induced by the tumor, thus allowing the activation of the anti-tumor immune response, while the checkpoint inhibitor (shown here with an anti-PD-1 antibody) will bind to the PD-1 molecule (or other checkpoint molecule depending on the inhibitor), which is in this case expressed on the surface of activated immune cells (cytoxic T lymphocytes) and prevent ligation by tumor expressed PD-L1 which would otherwise lead to CTL inhibition. Therefore, this two-pronged approach both allows activation of the immune cells and sustains that activity long enough to support anti-tumor efficacy, even in distinct tumor types that are resistant to at least one checkpoint inhibitor.

1. A method for prophylaxis and/or therapy of cancer comprising administering to an individual in need thereof a β-blocker and an immune checkpoint inhibitor.

2. The method of claim 1, wherein the immune checkpoint inhibitor is selected from anti-programmed cell death protein 1 (PD-1) antibody or PD-1 binding fragment thereof, or an anti-PD-1 principal ligand (PD-L1) antibody or PD-L1 binding fragment thereof.

3. The method of claim 1, wherein the β-blocker comprises a nonselective β-blocker.

4. The method of claim 1, wherein the immune checkpoint inhibitor is selected from an anti-PD-1 antibody or PD-1 binding fragment thereof, or an anti-PD-L1 antibody or PD-L1 binding fragment thereof and wherein the β-blocker comprises a nonselective β-blocker.

5. The method of claim 4 wherein the cancer is selected from breast cancer, pancreatic cancer, and melanoma.

6. The method of claim 4 wherein the administering of the β-blocker and the immune checkpoint inhibitor have a greater than additive inhibition of growth of the cancer.

7. The method of claim 1, wherein the individual has a cancer that was treated with the checkpoint inhibitor without the β-blocker, wherein the cancer was resistant to the checkpoint inhibitor, and wherein the β-blocker and the immune checkpoint inhibitor are subsequently administered to the individual such that growth of the cancer is inhibited.

8. The method of claim 7, wherein the inhibited growth of the cancer comprises inhibiting an increase in tumor volume.

9. A method comprising selecting an individual who has a cancer that exhibits resistance to treatment with a checkpoint inhibitor, wherein the individual is not treated with a β-blocker when the resistance is exhibited, and administering to the individual the checkpoint inhibitor and the β-blocker such that growth of the cancer is inhibited.

10. The method of claim 9, wherein the immune checkpoint inhibitor is selected from an anti-PD-1 antibody or PD-1 binding fragment thereof, or an anti-PD-L1 antibody or PD-L1 binding fragment thereof.

11. The method of claim 9, wherein the β-blocker comprises a nonselective β-blocker.

12. The method of claim 9, wherein the immune checkpoint inhibitor is selected from an anti-PD-1 antibody or PD-1 binding fragment thereof, or an anti-PD-L1 antibody or PD-L1 binding fragment thereof and wherein the β-blocker comprises a nonselective β-blocker.

13. The method of claim 12 wherein the cancer is selected from breast cancer, pancreatic cancer, and melanoma.

14. The method of claim 9, wherein the inhibited growth of the cancer comprises inhibiting an increase in tumor volume.

15. The method of claim 14 wherein the administering of the β-blocker and the immune checkpoint inhibitor have a greater than additive inhibition of increase in the tumor volume.
